(19) United States
(12) Patent Application Publication
(10) Pub. No.: US 2012/0177703 A1
(43) Pub. Date: Jul. 12, 2012

(54) SOLID LINEAR OLIGO-OR POLY-E-CAPROLACTONE

(76) Inventors: George Mihov, Maastricht (NL); Guy Draaisma, Papenhoven (NL)

(21) Appl. No.: 13/382,732

(22) PCT Filed: Jul. 9, 2010
(86) PCT No.: PCT/EP2010/059901

§ 371 Date: Mar. 26, 2012

(30) Foreign Application Priority Data
Jul. 9, 2009 (EP) 09165069.7
Apr. 6, 2010 (EP) PCT/EP2010/054518

(51) Int. Cl. A61K 47/34; A61P 27/02; A61K 9/14; C08G 63/66; A61K 9/00

(52) U.S. Cl. 424/400; 528/354; 514/772.7

(57) ABSTRACT

The present invention relates to linear oligo- or poly-ε-caprolactones di-blockcopolymers, in solid form at room temperature, comprising monoalkyl oligoethyleneglycol residues. The present invention further relates to a drug delivery formulation comprising above materials and a process for the preparation of the drug delivery formulation. The oligoethyleneglycol residues are preferably selected from the group consisting of methyl diethylene glycol, methyl triethyleneglycol or methyl tetraethylene glycol. The oligo- or poly-ε-caprolactone derivatives are prepared via the reaction of mono-hydroxy-oligoethyleneglycol with ε-caprolactone, whereby the mono-hydroxy-oligoethyleneglycol acts as an initiator.

The present invention relates to a solid material at room temperature comprising oligo- or poly-ε-caprolactones di-blockcopolymers, containing monoalkyl oligoethyleneglycol residues. The present invention further relates to a drug delivery formulation comprising above materials and a process for the preparation of the drug delivery formulation.

In drug delivery there is a continuous need for new materials with certain properties on molecular weight, morphology, melting point and viscosity. There is a further need for biocompatible materials which allow an easy tuning of polarity, melting point, sharp phase transition rate and long term drug releasing properties. It is however difficult to find biocompatible prior art materials with the above listed properties.

Linear oligo- or poly-ε-caprolactones di blockcopolymers comprising monoalkyl oligoethyleneglycol residues in aqueous medium are known from Moon Suk Kim et. al.: “Preparation of methoxy poly(ethyleneglycol)-block-poly(caprolactone) via activated monomer mechanism and examination of micellar characterization”, polymer bulletin 55, 149-156 (2005). This publication discloses methoxy poly (ethyleneglycol)-poly (ε-caprolactone) di-block copolymers and micelles made thereof with core shell architecture in an aqueous medium. The micelles seem to be the potential carriers for drug delivery. This publication teaches the influence of the Mw of the MPEG and the PCL blocks on the critical micelle concentration. The Mw of the MPEG varies from 550-2000 g/mol and shows a decreasing diameter of the micelles with an increasing Mw of MPEG block.

A disadvantage of the micelles is that they will not provide a long term release because release properties are form dependent.

In the present invention it is the object to provide new biocompatible and biodegradable materials which can be used for the long term release of drugs in the human or animal body and especially in the eye.

Several diseases and conditions of the posterior segment of the eye threaten vision. Age-related macular degeneration (AMD), choroidal neovascularization (CNV), retinopathies (e.g., diabetic retinopathy, vitreoretinopathy), retinitis (e.g., cytomegalovirus (CMV) retinitis), uveitis, macular edema, glaucoma, and neuropathies are several examples. These, and other diseases, can be treated by injecting a drug into the eye. Such injections are typically manually performed using a conventional syringe and needle. In using such a syringe, the surgeon is required to pierce the eye tissue with the needle, hold the syringe steady, and actuate the syringe plunger to inject the drug into the eye. Tissue damage may occur due to an “unsteady” injection. Reflux of the drug may also occur when the needle is removed from the eye. When a drug is to be injected into the eye, it is desirable to minimize the number of injections.

There is thus a need for a sustained drug release system based on biocompatible and biodegradable materials which can be melted, injected in for example the eye and which after injection become solid at body temperature and at the same time control the delivery of drugs over a certain period of time.

It is therefore an object of the present invention to find materials in solid form at room temperature with specific properties in view of molecular weight, crystallinity, melting point and viscosity.

It is a more specific object of the present invention to find biocompatible and biodegradable materials in solid form at room temperature, which are crystalline, which have melting points in the range of 40-80° C. and which have a viscosity in the range from 1-500 mPa·s at melt temperature.

The object of the present invention has been achieved in that new solid materials at room temperature have been found comprising linear oligo- or poly-ε-caprolactones di-blockcopolymers, containing a monoalkyl oligoethyleneglycol residue with a molecular weight <550 g/mol.

Room temperature is here and hereafter defined as a temperature of 20° C.

Unexpectedly it has been found that this solid material at room temperature comprising the linear oligo- or poly-ε-caprolactones derivatives containing monoalkyl oligoethyleneglycol residues with a molecular weight below 550 g/mol are fulfilling the material requirements of biocompatibility, biodegradability, melting point, viscosity and especially the requirement of providing a long term release.

The linear oligo- or poly-ε-caprolactones diblockcopolymers according to the present invention are in solid form at room temperature, are biocompatible and biodegradable. The solid materials preferably have melting points in the range of 40-80° C. and a viscosity at melt temperature in the range of 1-500 mPa·s.

The linear oligo- or poly-ε-caprolactones diblockcopolymers can be sterilized and remain stable after sterilization. In addition it is quite easy to tune the polarity, bio-erosion, melting point, phase transition rate and drug release properties by introducing a variety of monoalkyl oligoethyleneglycol residues of different molecular weights and structure. It is also possible to further tune the properties of thelinear oligo- or poly-ε-caprolactones diblockcopolymers by the degree of polymerisation.

Examples of monoalkyl oligoethyleneglycols are mono alkyl diethyleneglycol, mono alkyl triethylene glycol, mono alkyl tetra-ethylene glycol or mono alkyl penta-ethyleneglycol. The alkyl residue is selected from the group consisting of linear or branched alkyl comprising between 1-10 carbon atoms. Examples of such linear or branched alkyls are methyl, ethyl, propyl, isopropyl butyl or isobutyl. Preferred alkyl is methyl or ethyl. Preferred monoalkyl oligoethyleneglycol are methyldiethylene glycol, methyltriethylene glycol or methyl tetra-ethylene glycol.

The monoalkyl oligoethyleneglycol residue has a molecular weight <550, preferably below 455 g/mol, more preferably below 400 g/mol, most preferably below 250 g/mol. The Mw is measured by DSC, 3 minutes isotherm at 20° C., then 10° C. per minute scan rate, heated to 80° C. It has been found that the oligo- or poly-ε-caprolactones derivatives containing monoalkyl oligoethyleneglycol residues with a molecular weight below 550 g/mol will still remain sufficient hydrophobic to provide a long term release of for example hydrophobic bioactive agents. Moreover these materials do not have the tendency to swell which is important in case of intraocular injection. In case that the monoalkyl oligoethyleneglycol residue has a Mw which is higher than 550, the material will increasingly swell which is not preferred in certain drug delivery applications such as intraocular injections.

The linear oligo- or poly-ε-caprolactones diblockcopolymers preferably have a viscosity at melt temperature between 1 to 500 mPa·s, preferably between 1 to 200 mPa·s, more preferably between 5 and 100 mPa·s, most preferably between 10-30 mPa·s at melt temperature.

The linear oligo- or poly-ε-caprolactones diblockcopolymers preferably have a melting point in the range of 45-65° C., more preferably in the range from 50-55° C.

The present invention further relates to the solid material at room temperature comprising a linear oligo- or poly-ε-caprolactones diblockcopolymer and a further biodegradable polymer.

The biodegradable polymer may be selected from a polymer and/or a copolymer and/or a block co-polymer selected from the group consisting of poly-lactic acid (PLA), poly-glycolic acid (PGA), poly-lactide-co-glycolide (PLGA), polyesters, polyesteramides, poly (ortho ester), poly(phosphazine), poly (phosphate ester), oligo- or poly-ε-caprolactone, polyethylene glycol (PEG), gelatin, collagen, poly(D,L-lysine) or derivatives and combinations thereof. The biodegradable polymer may also be selected from lysinediisocyanate (LDI) which is functionalized with lipids via amide, urea or urethane bonds. The acid functionality of the lysinediisocyanate is for example protected by a group selected from ethyl, propyl, butyl, oligo-ethylene oxide or polyethylene oxide.

The lipids can be chosen from saturated fatty alcohols, fatty amines, fatty acids, cholesterol or sterols. Examples of the saturated fatty alcohols are 1-dodecanol, 1-decanol and 1-tetradecanol. Examples of the fatty amines are 1-decanolamine, 1-dodecanamine, 1-tetradecanamine. Examples of the fatty acids are decanoic acid, 1-dodecanoic acid (lauric acid) and 1-tetradecanoic acid (myristic acid). Preferably the lipid is chosen from a saturated fatty alcohol, fatty amine or fatty acid comprising at least 10 carbon atoms. More preferably the saturated fatty alcohol, fatty amine or fatty acid comprises from 12-14 carbon atoms. Preferably the biodegradable polymer is selected from the lysinediisocyanate (LDI) which is functionalized with lipids.

Unexpected it has been found that by blending the oligo- or poly-ε-caprolactones diblockcopolymers according to the present invention with a further biodegradable polymer, the biodegradability of the blend is improved compared to the biodegradability of the individual compounds. This means that the biodegradability of the oligo- or poly-ε-caprolactones diblockcopolymers can be influenced or tuned by blending it with a further biodegradable polymer. In drug delivery it is important that the biodegradability of the oligo- or poly-ε-caprolactones diblockcopolymers can be tuned easily. For some drug delivery applications one might need oligo- or poly-ε-caprolactones diblockcopolymers with different rates of biodegradation.

The amount of linear oligo- or poly-ε-caprolactones diblockcopolymers may vary from 10-90 weight %, the amount of biodegradable polymer may vary from 90-10 weight % based on the total weight of the composition. Preferably the amount of linear oligo- or poly-ε-caprolactones diblockcopolymers varies from 30-70 weight % and the amount of biodegradable polymer varies from 70-30 weight % based on the total weight of the composition. It is clear that the amount of linear oligo- or poly-ε-caprolactones diblockcopolymers and the amount of biodegradable polymer can be chosen such that the composition fulfils the above requirements of low viscosity in the melt, sharp phase transition and a melting point from 40-80° C.

The blends of the present invention can advantageously be used in drug delivery devices such as injection devices for ophthalmology. It is moreover possible to load the drug delivery devices with particles, capsules or nanospheres. The form of the particles, capsules or nanospheres may vary from porous, hollow, coated, or uncoated forms.

The solid material at room temperature according to the present invention may also comprise further biocompatible additives or surfactants. Non-limiting examples of biocompatible surfactants are polyoxamers and polysorbates.

If used in for example ophthalmology it is furthermore of importance that the diblockcopolymers are bioerodible, which means that they should break down over a prolonged period of time in response to the environment in the eye by one or more physical or chemical degradative processes, such as, enzymatic action, hydrolysis, ion exchange, dissolution by solubilization or emulsion formation. Likewise, the term “bioerode” is defined as the method by which such disintegration takes place. Bioerosion serves two purposes; it will release the bioactive agent at a controlled rate, but also it prevents remaining blockcopolymer in the tissues of the ocular cavity.

The oligo- or poly-ε-caprolactone diblockcopolymers according to the present invention are prepared by the reaction of mono-hydroxy-oligoethyleneglycol with ε-caprolactone. The mono-hydroxy-oligoethyleneglycol is acting as an initiator to start polymerization. An example of this reaction is given below:

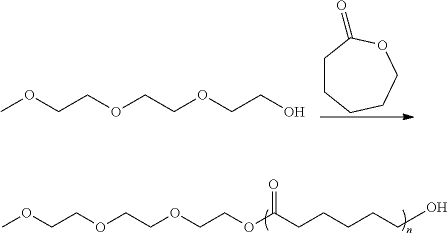

whereby n ranges from 5 to 50, preferably from 7 to 42.

The physical properties of the oligo- or poly-ε-caprolactone diblockcopolymers such as melting point, viscosity at melt temperature, biocompatibility and the hydrophilic properties can easily be tuned by the degree of polymerization.

The molecular weight of the oligo- or poly-ε-caprolactone diblockcopolymers for example ranges from 500-10.000 g/mol, preferably ranges from 1000-5000 g/mol, more preferably ranges from 1200-1900 g/mol.

The present invention further relates to a drug delivery formulation comprising a bioactive agent and at least one oligo- or poly-ε-caprolactone diblockcopolymer according to the present invention. Unexpectedly it has been found that drug delivery formulations can be provided which show a long term release. By long term release is meant a release of bioactive agent for at least one month, preferably at least 2 months, more preferably at least 3 months.

Examples of bioactive agents are nutrients, pharmaceuticals, proteins and peptides, vaccines, genetic materials, (such as polynucleotides, oligonucleotides, plasmids, DNA and RNA), diagnostic agents, and imaging agents. The bioactive agent may also be chosen from growth factors (VEGF, FGF, MCP-1, PIGF, antibiotics (for instance penicillin's such as B-lactams, chloramphenicol), anti-inflammatory compounds, antithrombogenic compounds, anti-claudication drugs, anti-arrhythmic drugs, anti-atherosclerotic drugs, anti-proliferatives, antihistamines, cancer drugs, vascular drugs, ophthalmic drugs, amino acids, vitamins, hormones, neurotransmitters, neurohormones, enzymes, signalling molecules and psychoactive medicaments.

Examples of specific bioactive agents or drugs are neurological drugs (amphetamine, methylphenidate), alpha1 adrenoceptor antagonist (prazosin, terazosin, doxazosin, ketenserin, urapidil), alpha2 blockers (arginine, nitroglycerin), hypotensive (clonidine, methyldopa, moxonidine, hydralazine minoxidil), bradykinin, angiotensin receptor blockers (benazepril, captopril, cilazepril, enalapril, fosinopril, lisinopril, perindopril, quinapril, ramipril, trandolapril, zofenopril), angiotensin-1 blockers (candesartan, eprosartan, irbesartan, losartan, telmisartan, valsartan), endopeptidase (omapatrilate), beta2 agonists (acebutolol, atenolol, bisoprolol, celiprolol, esmodol, metoprolol, nebivolol, betaxolol), beta2 blockers (carvedilol, labetalol, oxprenolol, pindolol, propanolol) diuretic actives (chlortalidon, chlorothiazide, epitizide, hydrochlorthiazide, indapamide, amiloride, triamterene), calcium channel blockers (amlodipin, barnidipin, diltiazem, felodipin, isradipin, lacidipin, lercanidipin, nicardipin, nifedipin, nimodipin, nitrendipin, verapamil), anti arthymic active (amiodarone, solatol, diclofenac, enalapril, flecamide) or ciprofloxacin, latanoprost, flucloxacillin, rapamycin and analogues and limus derivatives, paclitaxel, taxol, cyclosporine, heparin, corticosteroids (triamcinolone acetonide, dexamethasone, fluocinolone acetonide), anti-angiogenic (iRNA, VEGF antagonists: bevacizumab, ranibizumab, pegaptanib), growth factor, zinc finger transcription factor, triclosan, insulin, salbutamol, oestrogen, norcantharidin, microlidil analogues, prostaglandins, statins, chondroitinase, diketopiperazines, macrocycli compounds, neuregulins, osteopontin, alkaloids, immuno suppressants, antibodies, avidin, biotin, clonazepam.

Examples of specific ophthalmic bioactive agents are idoxuridine, phenylephrine, pilocarpine, eserine, carbachol, phospholine iodine, demecarium bromide, cyclopentolate, homatropine, scopolamine, epinephrine, hydrocortisone, hydrocortisone acetate, dexamethasone, dexamethasone prednisole 21-phosphate, prednisolone acetate, fluorometholone, beta-methasone, triamicinolone or antibiotics selected from the group consisting of tetracycline, chlorotetracycline, bacitracin, neomycin, polymyxin, gramicidin, oxytetracycline, chloramphenicol, gentamycin, penicillin and erythromycin.

The drug delivery formulation according to the present invention preferably comprises a polycaprolactone-triethyleneglycol diblockcopolymer with a Mw between 1000-5000 g/mol.

The present invention further relates to a process for the preparation of the drug delivery formulation comprising the steps of melting the oligo- or poly-ε-caprolactone diblockcopolymer containing a monoalkyl oligoethyleneglycol residue, mixing the melt with a bioactive agent and molding the mixture in a form. The oligo- or poly-ε-caprolactone diblockcopolymer is melted at temperatures between 40 and 80° C. The melt is mixed with from 1-40 weight % of bioactive agent based on the total weight of the formulation and molded into for example tablets, particles, spheres or rods. Preferably the melt is mixed with from 5-35 weight % of bioactive agent based on the total weight of the formulation. The resulting tablets, particles, spheres or rods can be used in medical devices for drug delivery such as injection devices. When used in injection devices the tablet or particle is melted just before injection and will solidify at body temperature after injection. It is of course also possible to extrude the solid materials according to the present invention.

Furthermore, the particles or tablets can be incorporated in for example (rapid prototyped) scaffolds, coatings, patches, composite materials, gels or plasters. The particles or tablets can also be sprayed, implanted or absorbed. Particles such as microparticles, nanoparticles are generally accepted as spherical particles with average diameters ranging from approximately 10 nm to 1000 micrometers. The preferred average diameter depends on the intended use. For instance, in case the particles are intended for use as an injectable drug delivery system, in particular as an intravascular drug delivery system, an average diameter of up to 10 μm, in particular of 1 to 10 μm may be desired. It is envisaged that particles with an average diameter of less than 800 nm, in particular of 500 nm or less, are useful for intracellular purposes. In other applications, larger dimensions may be desirable, for instance a diameter in the range of 1-100 μm or 10-100 μm. In particular, the particle diameter as used herein is the diameter as determinable by a LST 230 Series Laser Diffraction Particle size analyzer (Beckman Coulter), making use of a UHMW-PE (0.02-0.04 μm) as a standard. Particle-size distributions are estimated from Fraunhofer diffraction data and given in volume (%). If the particles are too small or non analyzable by light scattering because of their optical properties then scanning electron microscopy (SEM) or transmission electron microscopy (TEM) can be used.

The present invention further relates to coatings and implantable devices comprising the oligo- or poly-ε-caprolactone diblockcopolymers or the drug delivery formulation according to the present invention.

The invention will now be illustrated by the following examples without being limited thereto.

### Methods

Melting points were measured by accurately weighting the materials and place them in aluminum pans. Thermal behavior was assessed using a Mettler 822e heat-flux differential scanning calorimeter using the following method:

−20° C. to 85° C. at 10° C./min, isotherm for 3 min (heat 1)

85° C. to −20° C. at 10° C./min, isotherm for 3 min (cool)

−20° C. to 85° C. at 10° C./min, isotherm for 3 min (heat 2)

Viscosity measurements were performed on molten samples on the Physica MCR501-1 rheometer using the DoubleGAP geometry (DG26, 7 with 26.66 diameter and 7 μm concentricity).

### EXAMPLE 1

### PCL Synthesis-triethyleneglycolmonomethylether (TEGMME) Initiated Synthesis of PCL-1600-TEGMME

10.81 g triethyleneglycolmonomethylether was weight in a 250 ml round bottomed flask along with 90.04 g c-caprolactone. 329 mg. The flask was brought under an inert atmosphere and heated to 150° C. and stirred until a homogene mixture was formed. At this point 1 mL of a freshly prepared Sn(II)octoate catalyst solution in hexane was added (C_{(catalyst)}=33 g/L). Continued stirring and heating for an additional 16 hours to allow the reaction to proceed. The reaction mixture was cooled to room temperature after completion. The material was used as such without the need for additional purification.

1H-NMR samples were prepared in CDCl₃, no significant impurities were detected,

Mw (NMR)=1590 g/mol.
Melting point: 51.2° C. (1590 g/mol)

### EXAMPLE 2

### Release of Fluorescein and Disperse Red from PCL-1600-TEGMME

Test articles were prepared by mixing a UV-VIS absorbing dye into a melt of PCL1600-TEGMME, which was synthesized in example 1 and then cooled to solidify. As a dye, Fluorescein or Disperse Red were used and mixed with the material in a range of 0.5 to 30 wt % of the total mass.

The resulting tablets of approximately 12 mg weight were placed in 1 milliliter of phosphate buffered saline (for fluorescein containing materials) and in phosphate buffered saline with 0.4% sodium dodecyl sulphate (for Disperse Red containing materials). At given time points, the buffer solution was removed and measured for dye content by UV spectroscopy. The tablets were placed in fresh buffer solution until the next time point for exchange.

The resulting release curves are provided in FIGS. 1 and 2.

### EXAMPLE 3

### Release of a Bioactive Agent from PCL-1600-TEGMME

Tablets were prepared by mixing a bioactive agent into a melt of PCL-1600-TEGMME where after the mixture was cooled in a mould to solidify. The ratio of the therapeutic agent to PCL-1600-TEGMME mass was 10% by weight. The resulting tablets of approximately 12 mg weight were placed in 5 milliliter of phosphate buffered saline and shaked at 100 rpm and 37° C. At specific time points the buffer is fully exchanged. A volume of 900 μl buffer is mixed with 600 μl acetonitril in a HPLC vial and analyzed by HPLC to determine the drug concentration.

The resulting release curve is provided in FIG. 3.

### EXAMPLE 4

### In Vitro Erosion of PCL-1600-TEGMME

In a continuous-flow system with a flow rate of 7.5 ml/min of PBS at 37° C., weight loss of tablets of PCL-1600-TEGMME was observed. Hereto, 10 tablets were placed in a glass filter (diameter 10 mm, porosity 1) and gentle flow was applied from the bottom. A volume of 1 liter PBS was re-circulated for one week upon which the samples were removed from the glass filter, rinsed in DI water three times and dried under vacuum at 30° C. for 40 hrs. Weight was recorded and samples were again transferred to the flow system for erosion testing. The resulting graph is provided in FIG. 4.

### EXAMPLE 5

### Steady Shear Rate Sweep and Temperature Sweep of PCL1300-TEGMME

The viscosity of PCL1300-TEGMME was measured using molten samples on a Physica MCR501-1 rheometer using the DoubleGAP geometry (DG26.7 with 26.66 diameter and 7 μm concentricity). The following measurements were performed:
Steady rate sweep at 65° C. (0.1-10000 s⁻¹).
Next temperature sweeps from 65-30 and back from 30-65° C. (in oscillation with 0.1% strain and frequency of 10 rad/s and cooling/heating rate of 1° C. min⁻¹).
   Results are shown in FIGS. 5 and 6.

### EXAMPLE 6

### Preparation of a PCL-TEG/C12-LDI Blend

0.9 g of PCL-TEG and 2.1 g C12-LDI were weight into one sample vial and heated to 80° C. until the substances were molten. The melt was mixed on a vortex for 1 minute and poured into a mold where it solidified.

### EXAMPLE 7

2.1 g of PCL-TEG and 0.9 g C12-LDI were weight into one sample vial and heated to 80° C. until the substances were molten. The melt was mixed on a vortex for 1 minute and poured into a mold where it solidified.

### EXAMPLE 8

### In Vitro Erosion Test of PCL-TEG/C12-LDI Blend

The in vitro flow erosion of the pure substances of C12-LDI and PCL-TEG and blends of the pure substances in the ratio 30/70 (w %/w %) and 70/30 (w %/w %) were measured. Pure C12-LDI is eroding very slowly compared to PCL-TEG. PCL-TEG lost 28 w % after 70 days while C12-LDI lost only 3 w % after the same time. Blends of the 2 substances show erosion in between the pure materials and respective to the PCL-TEG content.

Results are shown in FIG. 7.

### DESCRIPTION OF THE FIGURES

FIG. 1: Average release of fluorescein (n=3) in PCL1600-TEGMME

FIG. 2: Average release of Disperse Red (n=3) in PCL1600-TEGMME

FIG. 3: Release of a bioactive agent from PCL1600-TEGMME

FIG. 4: In vitro erosion of PCL-1600-TEGMME

FIG. 5: Steady Shear Rate Sweep of PCL1300-TEGMME

FIG. 6: Dynamic viscosity and crystallization and melting in temperature sweep of PCL1300-TEGMME

FIG. 7: In vitro erosion test of PCL-TEG/C12-LDI blend

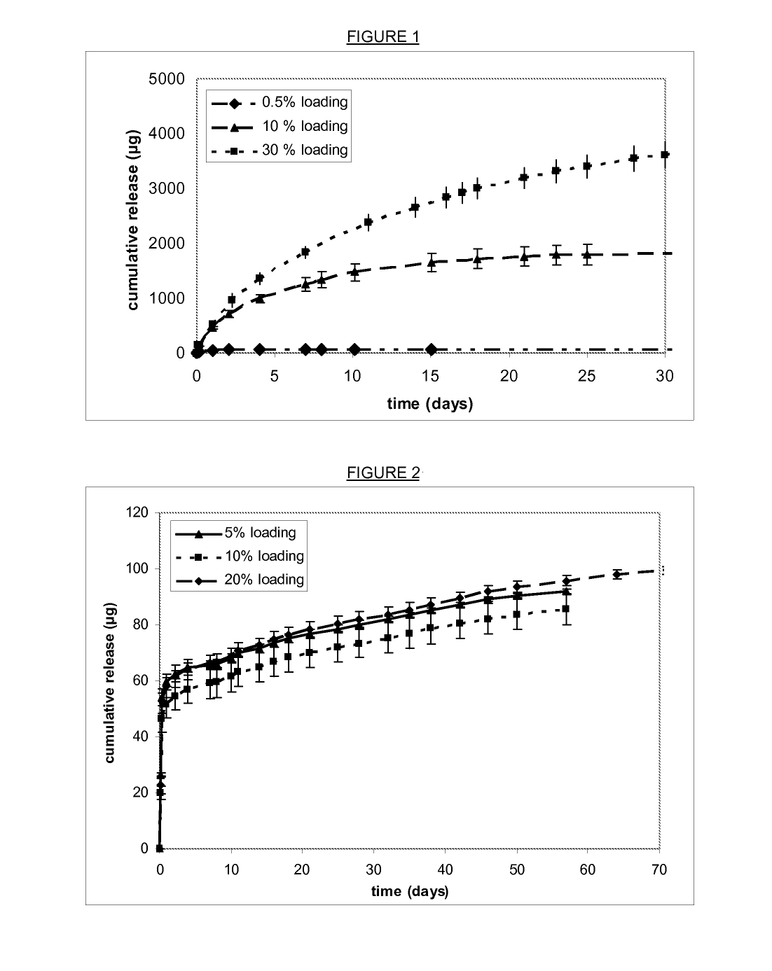

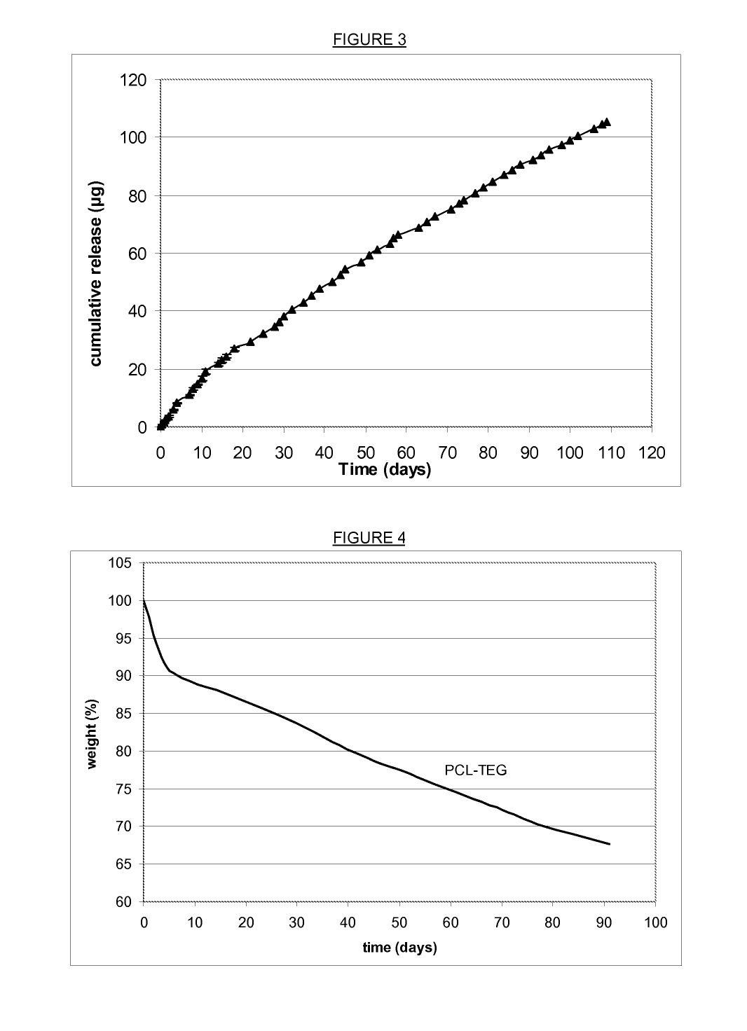

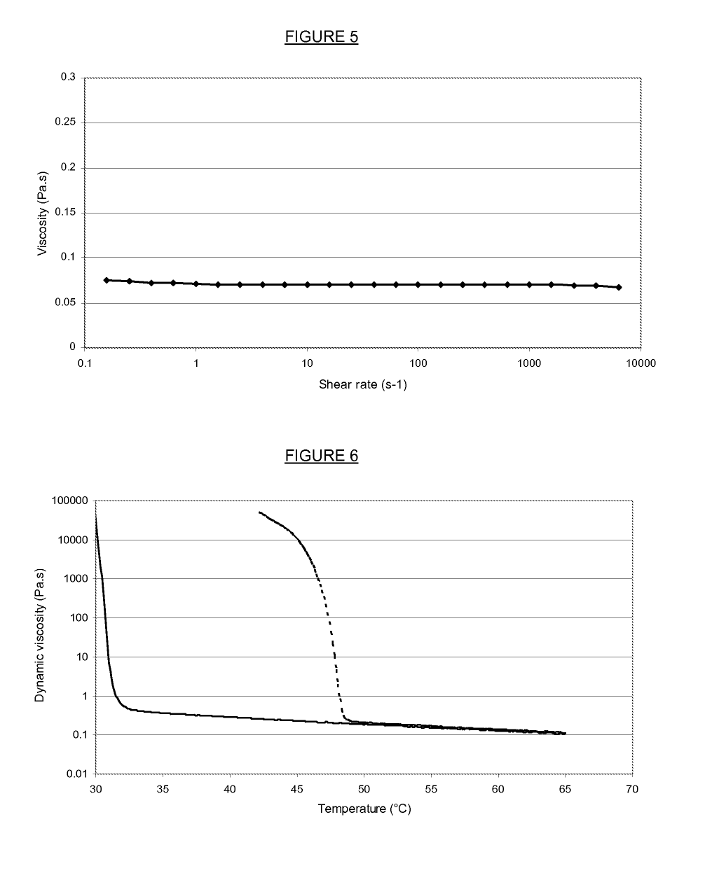

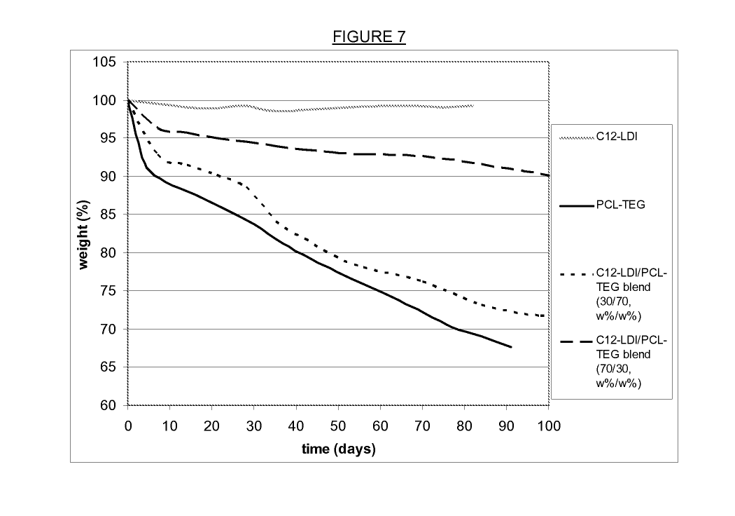

1. Solid material at room temperature comprising linear oligo- or poly-ε-caprolactone diblockcopolymer containing a monoalkyl oligoethyleneglycol residue with a molecular weight <550 g/mol measured by DSC.

2. Solid material at room temperature comprising linear oligo- or poly-ε-caprolactone diblockcopolymers according to claim 1 wherein the molecular weight of the monoalkyl oligoethyleneglycol <455 g/mol.

3. Solid material at room temperature comprising linear oligo- or poly-ε-caprolactone diblockcopolymers according to claim 1 wherein the monoalkyl oligoethyleneglycol residue is selected from the group consisting of methyl diethylene glycol, methyl triethyleneglycol or methyl tetra-ethylene glycol.

4. Solid material at room temperature comprising linear oligo- or poly-ε-caprolactone diblockcopolymers according to claim 1 having a melting point between 40 and 80° C.

5. Solid material at room temperature comprising linear oligo- or poly-ε-caprolactone diblockcopolymers according to claim 1 having a viscosity at melt temperature between 1 to 500 mPa·s.

6. Solid material at room temperature comprising linear oligo- or poly-ε-caprolactone diblockcopolymers according to claim 1 further comprising a biodegradable polymer.

7. Solid material at room temperature comprising linear oligo- or poly-ε-caprolactone diblockcopolymers according to claim 1 wherein the biodegradable polymer is selected from the group consisting of poly-lactic acid (PLA), poly-glycolic acid (PGA), poly-lactide-co-glycolide (PLGA), polyesters, polyesteramides, poly (ortho ester), poly(phosphazine), poly (phosphate ester), polyethylene glycol (PEG), gelatin, collagen, poly(D,L-lysine) or derivatives and combinations thereof.

8. Drug delivery formulation comprising a bioactive agent and at least one oligo- or poly-ε-caprolactone diblockcopolymer according to claim 1.

9. Drug delivery formulation according to claim 8 wherein the bioactive agent is an ophthalmic bioactive agent.

10. Drug delivery formulation according to claim 8 wherein the oligo- or poly-ε-caprolactone diblockcopolymer is a polycaprolactone-triethyleneglycol diblockcopolymer with a Mw between 1000-5000 g/mol.

11. Process for the preparation of the drug delivery formulation according to claim 8 comprising the steps of melting the oligo- or poly-ε-caprolactone diblockcopolymer containing a monoalkyl oligoethyleneglycol residue, mixing the melt with a bioactive agent and molding the mixture in a form.

12. Process according to claim 11 wherein the form is a tablet, rod, sphere or particle.

13. Use of the drug delivery formulation according to claim 8 in an injectable device for delivery of drugs in the eye.

14. Coatings comprising the oligo- or poly-ε-caprolactone diblockcopolymers according to claim 1.

15. Implantable devices comprising the oligo- or poly-ε-caprolactone diblockcopolymers according to claim 1.
